(19) United States
(12) Patent Application Publication
(10) Pub. No.: US 2014/0324146 A1
(43) Pub. Date: Oct. 30, 2014

(54) STAND-ALONE ELECTRONIC APPARATUS AND DEVICE FOR FASTENING THE LATTER TO AN ELECTRODE LOCATED ON THE HEAD OF A PATIENT FOR USE WHEN THE PATIENT IS LYING DOWN

(71) Applicant: Pierre-Yves Muller, Collonge-bellerive (CH)

(72) Inventor: Pierre-Yves Muller, Collonge-bellerive (CH)

(73) Assignee: STX-Med Sprl, Liege (BE)

(21) Appl. No.: 14/328,396

(22) Filed: Jul. 10, 2014

Related U.S. Application Data
(63) Continuation of application No. PCT/EP2013/050047, filed on Jan. 3, 2013.

(30) Foreign Application Priority Data
Jan. 13, 2012 (BE) 2012/0025

(51) Int. Cl. A61N 1/04

(52) U.S. Cl. CPC A61N 1/0492; A61N 1/0456; A61N 1/048
USPC 607/139

(57) ABSTRACT

Stand-alone apparatus intended to be fastened on to a body part, comprising a bipolar electrode (1), a light electronic device (2), means for the mechanical fastening of the electronic apparatus (2) to the electrode (1) and means enabling the electrical current to flow from the electronic apparatus (2) to the electrode (1) and vice versa. Said fastening means comprise two permanent magnets (4) located on the electronic apparatus (2), each of which is electrically conductive and capable of cooperating with a magnetisable metal zone (3) located on the side of the electrode (1) opposite the side intended to be in contact with the skin.

### CROSS-REFERENCE TO RELATED PATENT APPLICATIONS

This patent application is a continuation of PCT/EP2013/050047, filed Jan. 3, 2013, which claims priority to Belguim Application No. 2012/0025 , filed Jan. 13, 2012, the entire teachings and disclosure of which are incorporated herein by reference thereto.

### SUBJECT MATTER OF THE INVENTION

The present invention relates to the field of apparatuses either supplying electrical impulses to the body, in particular to the cranium, for the purposes of neurostimulation, or receiving electrical impulses by way of biofeedback.

### STATE OF THE ART

Neurostimulation is a well-known technique that consists in using electrical impulses to generate action potentials on nerves.

It has recently been demonstrated that the stimulation of the upper branch of the trigeminal nerve had a sedative effect in humans (Supraorbital transcutaneous neurostimulation has sedative effects in healthy subjects, Piquet M., Balestra C., Sava S. L., Schoenen J. E., BMC Neurology 2011, 11 :135). This effect could be useful for patients suffering from insomnia. The corresponding therapeutic method is the subject matter of patent application no U.S. Ser. No. 13/101,495 (published under the number US 2011/0282129).

A problem that arises in the application of this method is that the neurostimulation apparatuses according to the state of the art always contain cables providing for the connection of the electronic apparatus to the electrode in contact with the skin. These cables clearly act as a brake on the use in any application relating to sleep. They indeed run the risk of easily getting wrapped around the patient and detaching the electrode. They thus require the patient to maintain a stable position during sleep, which is not the case if their sleep is somewhat agitated.

Recently the company STX-Med (Herstal, Belgium) has developed an apparatus for stimulating the trigeminal nerve on the frontal part of the cranium (Apparatus for electro-inhibition of facial muscles, WO 2006/063417 ; Device for the electrotherapeutic treatment of tension headaches, US 2009/0210028). This stand alone apparatus does not use cables, but rather lateral arms in a manner similar to eye glasses for positioning the electrode on the forehead. However, these arms constitute, in the same way as cables, an inconvenience for patients lying down.

Document U.S. Pat. No. 4,112,941 discloses the assembly of an improved biomedical electrode and a connector. The latter comprises a magnet facilitating the coupling with a projecting portion of the electrode. The electrical contact between the connector and the electrode occurs along the projecting portion in a manner so as to form a space between the connector and the flat surface of the electrode. This space is used to loosen the assembly to some extent.

### AIMS OF THE INVENTION

The invention seeks to enable the fastening of a stand-alone electronic apparatus of small size on the body of a patient, in particular on the cranium, in applications that require the patient to be lying down.

In particular, the aim of the invention is to enable the stimulation of the upper branch of the trigeminal nerve in a manner that is comfortable, simple and compatible with a use during sleep.

### MAIN FEATURES OF THE INVENTION

A first aspect of the present invention relates to a stand-alone apparatus designed to be fastened on to a body part, comprising a bipolar electrode with a flat support element provided on one side with a conductive self-adhesive gel for the adhesion to the skin, a light electronic device capable of generating or receiving electrical currents respectively to or from the body, through the electrode, means for the mechanical fastening of the electronic apparatus to the electrode and means for making the electrical current flow from the electronic apparatus to the electrode and vice versa, characterised in that said fastening means comprise two permanent magnets located on the electronic apparatus, each of which is electrically conductive and capable of cooperating with a magnetisable metal zone located on the side of the electrode opposite the side intended to be in contact with the skin.

According to preferred embodiments, the apparatus according to the invention further comprises one or a suitable combination of the following features:

the magnetisable metal zone consists of a ferromagnetic surface;
the ferromagnetic surface contains iron, cobalt, nickel and/or one of the alloys thereof;
the two permanent magnets are electrically conductive or coated with an electrical conductor that enables the flow of current between the apparatus and the electrode;
each permanent magnet is a rare earth alloy magnet;
the rare earth alloy magnet is a neodymium-iron-boron magnet;
the rare earth alloy magnet is a samarium-cobalt magnet ;
the rare earth alloy magnet is coated with a layer of nickel, copper, zinc, chromium, gold, silver or epoxy resin;
the rare earth alloy magnet has a maximum energy product BHₘₐₓ comprised between 30 and 52 MGOe;
each permanent magnet is cylindrical with an axis perpendicular to the surface of the apparatus intended to come into contact with the electrode ;
the electronic apparatus has a casing that is flat and in the approximate shape of a diamond;
the electrode comprises two circular magnetisable zones whose respective centres have the same spacing as the respective centres of the two magnets located on the electronic apparatus, on its side intended to make contact with the electrode.

Another aspect of the present invention is related to the use of the above mentioned apparatus for the stimulation of supraorbital and supratrochlear afferents of the upper branch of the trigeminal nerve, for a recording of bioelectric activity in a biofeedback application or, more generally, in the context of an application where the patient is lying down or for an application during sleep.

### BRIEF DESCRIPTION OF THE FIGURES

FIG. 1 shows an embodiment of a stand-alone electronic apparatus for front fastening on the head, according to the present invention.

FIG. 2 shows an embodiment of the electrode suitable for the fastening of the electronic apparatus according to FIG. 1.

### DETAILED DESCRIPTION OF THE INVENTION

The object of the invention is an assembly consisting of a stand-alone electronic device, that is, without wires or cables and powered by means of a rechargeable battery, and an electrode enabling it to be fastened to a body part, preferably the head, more preferably the forehead, in a manner so as to either stimulate the muscles and/or nerves, or to record the bioelectrical activity of the patient.

The main features of this invention are as follows:

the use of an electrode, with a conductive self-adhesive gel electrode on its side intended to come into contact with the skin, which holds the system on the forehead with an electrical contact,
the use of magnets which enable the following:

holding the apparatus on the electrode, in contact with the latter

easily and quickly positioning the apparatus on the electrode, even in the dark ;

transmitting the electrical currents between the apparatus and the electrode, as long as the magnets used are sufficiently electrically conductive.

### The Electrode

The electrode according to the invention is a bipolar electrode whose dimensions are selected so as to be comfortable to use and in order to stimulate the supraorbital and supratrochlear afferents of the upper branch of the trigeminal nerve (for a mode of execution with respect to preferred shape and dimensions, see US 2009/0210028). It comprises, on its internal side, a layer of self-adhesive gel that enables an autonomous adhesion to the cranium skin and, on its external side, a magnetisable metal portion (for example made of soft iron).

### Electronic Apparatus

The apparatus according to the invention is provided with at least two powerful permanent magnets, preferably two magnets, which enable the following:

holding the apparatus on the electrode, which itself adheres to the forehead. In this manner, the electrode-apparatus system is held in place permanently and allows a use by the patient while lying down,
easy positioning of the apparatus: the magnets are attracted to the magnetisable portions on the electrode and therefore position themselves spontaneously in the appropriate places. This allows to reposition the apparatus without needing any light (for example, during the night), or any mirror;
conducting the electrical currents, either from the apparatus to the body in the case of neurostimulation, or from the body to the apparatus in the case of biofeedback;
avoiding damage to the electrode during the positioning as well as during the removal of the apparatus : the connection by means of magnetisation avoids the mechanical stresses on the potentially fragile parts of an electrode;
not having any loose connections: once in position, the magnets remain immobile, contrary to a connection of the mechanical type that may shift during a movement of the head for example or that may become loose through repeated use.

The apparatus is designed in the chosen materials so as to be made very light, which avoids the exertion of an excessive stress on the electrode and prevents the latter from becoming detached.

The connection by means of magnets could also enable a facilitated connection in the use of a headband for occipital stimulation.

### DESCRIPTION OF A PREFERRED EMBODIMENT OF THE INVENTION

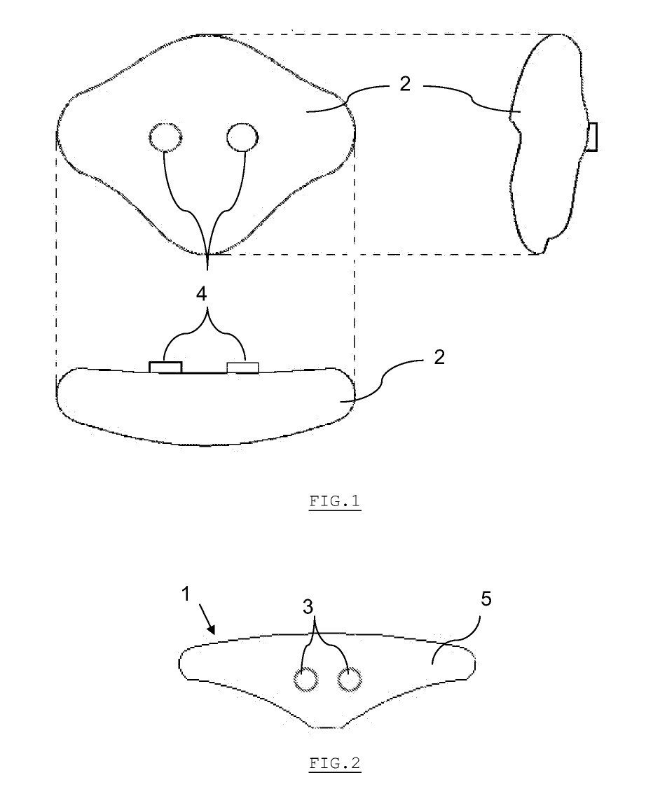

According to a preferred embodiment of the invention, the apparatus 2 has a general flattened diamond shape, as shown in FIG. 1. The two magnets 4 are advantageously of the sintered Neodymium NdFeB (neodymium-iron-boron) type. They may possibly be covered or coated with a metal layer, for example with nickel, copper, zinc, chromium, gold, silver, etc, or partially with epoxy resin, so as to ensure protection against corrosion or to improve the flow of the electrical current. They have an adhesive strength of the order of 250 g and a maximum energy product (BHₘₐₓ) of the order of 48 MGOe. The magnets 4 are cylindrical.

The electrode 1 is a self-adhesive gel bipolar electrode and has on its exterior side, as shown in FIG. 2, two circular magnetisable zones 3, the centres of which have the same spacing (or center-to-center distance) as the respective centres of the two magnets 4 on the apparatus 2.

### REFERENCE SYMBOLS

1. electrode
2. electronic apparatus
3. magnetisable zone
4. permanent magnet.

1. A stand-alone electronic apparatus designed to be fastened on to the forehead of a patient, comprising an elongated electrode (1) with a flat support element provided on one side with a conductive self-adhesive gel for the adhesion to the skin, a light and small electronic device (2) capable of generating or receiving electrical currents respectively to or from the body, through the electrode (1), means for fastening the electronic apparatus (2) to the electrode (1) in the form of permanent magnets (4) located on the electronic device (2), which are electrically conductive in themselves or coated with an electrical conductor and each being capable of cooperating with a magnetisable metal zone (3) located on the side of the electrode (1) opposite the side intended to be in contact with the skin, in order to make the electrical current flow directly from the electronic device (2) to the electrode (1) and vice versa, characterised in that the electrode (1) is bipolar and comprises two circular magnetisable zones (3) arranged along the longitudinal direction of the electrode and whose respective centres have the same spacing as the respective centres of the two magnets (4) located on the electronic apparatus (2), on its side intended to make contact with the electrode.

2. An apparatus according to claim 1, characterised in that the magnetisable metal zone (3) consists of a ferromagnetic surface.

3. An apparatus according to claim 1, characterised in that the ferromagnetic surface contains iron, cobalt, nickel and/or one of the alloys thereof.

4. An apparatus according to claim 1, characterised in that each permanent magnet is a rare earth alloy magnet.

5. An apparatus according to claim 4, characterised in that the rare earth alloy magnet is a neodymium-iron-boron magnet.

6. An apparatus according to claim 4, characterised in that the rare earth alloy magnet is a samarium-cobalt magnet.

7. An apparatus according to claim 4, characterised in that the rare earth alloy magnet is coated with a layer of nickel, copper, zinc, chromium, gold, silver or epoxy resin.

8. An apparatus according to claim 4, characterised in that the rare earth alloy magnet has a maximum energy product BHₘₐₓ comprised between 30 and 52 MGOe.

9. An apparatus according to claim 1, characterised in that each permanent magnet is cylindrical with an axis perpendicular to the surface of the electronic apparatus (2) intended to come into contact with the electrode (1).

10. An apparatus according to claim 1, characterised in that the electronic device (2) has a casing that is flat and in the approximate shape of a diamond.

11. Use of the apparatus according to claim 1, for the stimulation of supraorbital and supratrochlear afferents of the upper branch of the trigeminal nerve.

12. Use of the apparatus according to claim 1, for a recording of bioelectric activity in a biofeedback application.

13. Use of the apparatus according to claim 1, in the context of an application where the patient is lying down or for an application during sleep.
